(19) United States
(12) Patent Application Publication
(10) Pub. No.: US 2010/0076490 A1
(43) Pub. Date: Mar. 25, 2010

(54) FACET JOINT BROACHING INSTRUMENT, IMPLANT, AND ASSOCIATED METHOD

(76) Inventors: JONATHAN GREENWALD, Boca Raton, FL (US); Deanna Ally, Dania Beach, FL (US)

Correspondence Address: Clements Bernard PLLC, 1901 Roxborough Road, Suite 250, Charlotte, NC, 28211, US

(21) Appl. No.: 12/395,300

(22) Filed: Feb. 27, 2009

Related U.S. Application Data
(60) Provisional application No. 61/032,193, filed on Feb. 28, 2008.

(51) Int. Cl. A61B 17/88; A61B 17/70

(52) U.S. Cl. 606/279; 606/86 A

(57) ABSTRACT

The present disclosure relates to a facet joint broaching instrument, implant, and associated method for stabilizing/immobilizing a facet joint of the spine. The instrument is configured to bore a broaching spike through the facet joint to create a bore in the facet joint. The instrument is further configured to remove excess bone material from the broaching operation through the bore. Once a bore is formed through the facet joint, a facet joint implant is positioned through the bore thereby stabilizing and immobilizing the facet joint. In an exemplary embodiment, the instrument includes a pair of handles, a hinge, opposing arms, a broaching spike, and a receiving chamber for excess bone. The present invention further includes the implant for use with the instrument and an associated surgical method for stabilizing and immobilizing a facet joint with the implant and the instrument.

### CROSS-REFERENCE TO RELATED APPLICATION(S)

The present non-provisional utility patent application claims priority to U.S. Provisional Patent Application Ser. No. 61/032,193, filed Feb. 28, 2008, and entitled “FACET JOINT BROACHING INSTRUMENT, IMPLANT, AND ASSOCIATED METHOD,” the contents of which are herein incorporated by reference in-full.

### FIELD OF THE INVENTION

The present invention relates generally to systems and methods for stabilizing/immobilizing a portion of the spine during a surgical procedure. More specifically, the present invention relates to a facet joint broaching instrument, implant, and associated method for stabilizing/immobilizing a facet joint of the spine.

### BACKGROUND OF THE INVENTION

In the treatment of various spinal ailments/defects, it is desirable to stabilize/immobilize one or more facet joints of the spine, either via an open or minimally-invasive surgical procedure. There are numerous conventional instruments, implants, and associated methods for performing such stabilization/immobilization via numerous approaches. Bone arthrodesis, or fusion, is a surgical procedure that is used to stabilize or immobilize impaired bones or joints such that they can heal. More specifically, facet arthrodesis is a surgical procedure that is used to stabilize or immobilize a spinal facet joint in the treatment of an injury or degenerative condition. Conventional facet arthrodesis systems and methods utilize bone screws that are driven through the superior and inferior facets so as to allow the adjoined bone sections to fuse together. Conventional facet arthrodesis systems and methods also utilize wires that are looped around the superior and inferior facets so as to allow the adjoined bone sections to fuse together. The surgical procedures that must be employed to implant these bone screws or wires are difficult and time consuming. What is still needed in the art, however, is an improved instrument, implant, and associated method for performing such stabilization/immobilization that may be effectively utilized in only a few relatively simple steps, making it essentially foolproof for a surgeon employing it.

### BRIEF SUMMARY OF THE INVENTION

In various exemplary embodiments, the present invention relates to a facet joint broaching instrument, implant, and associated method for stabilizing/immobilizing a facet joint of the spine. The instrument is configured to bore a broaching spike through the facet joint to create a bore in the facet joint. The instrument is further configured to remove excess bone material from the broaching operation through the bore. Once a bore is formed through the facet joint, a facet joint implant is positioned through the bore thereby stabilizing and immobilizing the facet joint. In an exemplary embodiment, the instrument includes a pair of handles, a hinge, opposing arms, a broaching spike, and a receiving chamber for excess bone. The present invention further includes the implant for use with the instrument and an associated surgical method for stabilizing and immobilizing a facet joint with the implant and the instrument.

In one exemplary embodiment, the present invention provides a facet joint broaching instrument and implant for stabilizing/immobilizing a facet joint of the spine via an open or minimally-invasive surgical procedure, including: a broaching mechanism including a pair of opposed arms, wherein one of the pair of opposed arms is coupled to a broaching spike and the other of the pair of opposed arms defines a bore configured and positioned to receive the broaching spike; and an actuation mechanism operable for selectively compressing the pair of opposed arms of the broaching mechanism together and retracting the pair of opposed arms of the broaching mechanism apart. Optionally, the actuation mechanism includes a pair of handles connected via a hinge and coupled to the broaching mechanism. Optionally, the facet joint broaching instrument and implant also includes a pair of opposed plates coupled to the pair of opposed arms, wherein the pair of opposed plates are shaped and sized to securely engage a superior facet and an inferior facet of a facet joint of a spine. Optionally, the facet joint broaching instrument and implant further includes a receiving chamber coupled to the other of the pair of opposed arms, wherein the receiving chamber is configured and positioned to collect bone material that is broached from a facet joint of a spine and pushed through the bore by the broaching spike when the broaching mechanism is actuated. Preferably, the facet joint broaching instrument and implant still further includes, with or without the broaching spike removed, a facet joint broaching implant including a facet joint broaching bolt selectively engaging the one of the pair of opposed arms and a facet joint broaching nut selectively engaging the other of the pair of opposed arms. The facet joint broaching bolt includes a head portion and a shaft portion, wherein the head portion of the facet joint broaching bolt is configured to selectively engage the one of the pair of opposed arms and the shaft portion of the facet joint broaching bolt includes a plurality of raised structures that are configured to securely engage one or more raised structures disposed within a bore associated with the facet joint broaching nut, and wherein the facet joint broaching nut is configured to selectively engage the other of the pair of opposed arms. Optionally, the facet joint broaching nut includes a plurality of tooth structures configured to frictionally engage a surface of a facet joint of a spine.

In another exemplary embodiment, the present invention provides a facet joint broaching method for stabilizing/immobilizing a facet joint of the spine via an open or minimally-invasive surgical procedure, including: providing a broaching mechanism including a pair of opposed arms, wherein one of the pair of opposed arms is coupled to a broaching spike and the other of the pair of opposed arms defines a bore configured and positioned to receive the broaching spike; and providing an actuation mechanism operable for selectively compressing the pair of opposed arms of the broaching mechanism together and retracting the pair of opposed arms of the broaching mechanism apart. Optionally, the actuation mechanism includes a pair of handles connected via a hinge and coupled to the broaching mechanism. Optionally, the facet joint broaching method also includes providing a pair of opposed plates coupled to the pair of opposed arms, wherein the pair of opposed plates are shaped and sized to securely engage a superior facet and an inferior facet of a facet joint of a spine. Optionally, the facet joint broaching method further includes providing a receiving chamber coupled to the other of the pair of opposed arms, wherein the receiving chamber is configured and positioned to collect bone material that is broached from a facet joint of a spine and pushed through the bore by the broaching spike when the broaching mechanism is actuated. Preferably, the facet joint broaching method still further includes, with or without the broaching spike removed, providing a facet joint broaching implant including a facet joint broaching bolt selectively engaging the one of the pair of opposed arms and a facet joint broaching nut selectively engaging the other of the pair of opposed arms. The facet joint broaching bolt includes a head portion and a shaft portion, wherein the head portion of the facet joint broaching bolt is configured to selectively engage the one of the pair of opposed arms and the shaft portion of the facet joint broaching bolt includes a plurality of raised structures that are configured to securely engage one or more raised structures disposed within a bore associated with the facet joint broaching nut, and wherein the facet joint broaching nut is configured to selectively engage the other of the pair of opposed arms. Optionally, the facet joint broaching nut includes a plurality of tooth structures configured to frictionally engage a surface of a facet joint of a spine. Preferably, the broaching mechanism is selectively disposed about a facet joint of a spine through an access tube.

In yet another exemplary embodiment, a facet joint implantation method for stabilizing/immobilizing a facet joint of the spine via an open or minimally-invasive surgical procedure includes inserting an access tube normal to a facet joint of a patient; positioning a broaching instrument through the access tube adjacent to a superior and inferior facet of the facet joint; closing tongs on the broaching instrument to force a sharp pin to broach through the superior and inferior facet to create a hole; releasing the tongs; pulling the broaching instrument out of the tube; positioning an implant instrument in the access tube with an implant attached; closing tongs on the implant instrument to compress the implant through the hole previously broached; and removing the implant instrument through the access tube with the implant left through the facet joint. The facet joint implantation method further includes pushing excess bone material through the hole while the sharp pin is broached through the superior and inferior facet; and collecting the excess bone material in a receiving chamber of the broaching instrument. The facet joint implantation method further includes adjusting a nut of the implant to a desired position to lock the implant to the facet joint. The broaching instrument and the implant instrument can include a single instrument with the sharp pin, the receiving chamber, and the implant detachable from the single instrument.

### BRIEF DESCRIPTION OF THE DRAWINGS

The present invention is illustrated and described herein with reference to the various drawings, in which like reference numbers denote like method steps and/or system components, respectively, and in which:

FIG. 1 is a side view of one exemplary embodiment of the facet joint broaching instrument of the present invention;

FIG. 2 is an exploded perspective view of the broaching mechanism of the facet joint broaching instrument of FIG. 1 according to an exemplary embodiment of the present invention

FIG. 3 is an enlarged side view of a portion of the facet joint broaching instrument of FIG. 1, the facet joint broaching instrument engaging a facet joint of the spine;

FIG. 4 is an exploded side view of one exemplary embodiment of the facet joint broaching implant of the present invention, the facet joint broaching implant including a facet joint broaching bolt and a facet joint broaching nut;

FIG. 5 is an enlarged end view of the facet joint broaching nut of FIG. 3 according to an exemplary embodiment of the present invention;

FIG. 6 is a side view of one exemplary embodiment of the facet joint implant instrument of the present invention;

FIG. 7 is an exploded perspective view illustrates an engagement mechanism on the facet joint implant instrument of FIG. 6 according to an exemplary embodiment of the present invention.

FIG. 8 is a side view of the facet joint implant instrument of FIG. 6 engaging the facet joint;

FIG. 9 is a side view of the facet joint implant instrument of FIG. 6 disengaging the facet joint and with the facet joint broaching implant of FIG. 4 engaged to a bore formed in FIG. 6; and

FIG. 10 is a flow chart of an exemplary implantation method utilizing the facet joint broaching instrument of FIG. 1, the facet joint broaching implant of FIG. 4, and the facet joint implant instrument of FIG. 6.

### DETAILED DESCRIPTION OF THE INVENTION

In various exemplary embodiments, the present invention relates to a facet joint broaching instrument, implant, and associated method for stabilizing/immobilizing a facet joint of the spine. The instrument is configured to bore a broaching spike through the facet joint to create a bore in the facet joint. The instrument is further configured to remove excess bone material from the broaching operation through the bore. Once a bore is formed through the facet joint, a facet joint implant is positioned through the bore thereby stabilizing and immobilizing the facet joint. In an exemplary embodiment, the instrument includes a pair of handles, a hinge, opposing arms, a broaching spike, and a receiving chamber for excess bone. The present invention further includes the implant for use with the instrument and an associated surgical method for stabilizing and immobilizing a facet joint with the implant and the instrument.

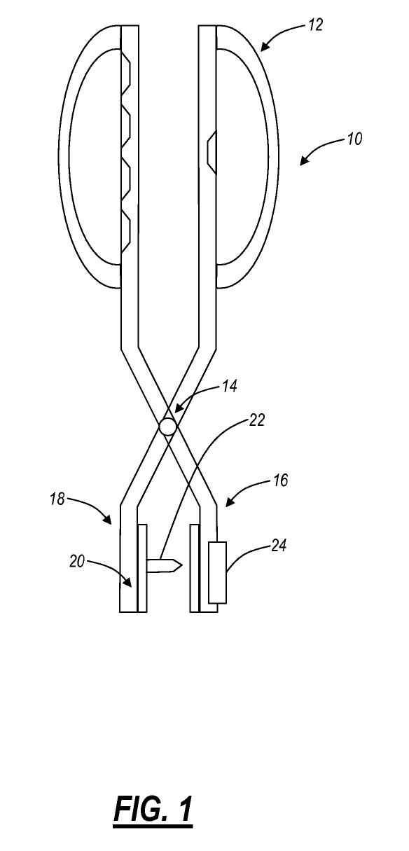

Referring to FIG. 1, a facet joint broaching instrument 10 includes a pair of handles 12 that are connected via a hinge 14 and operable for actuating a broaching mechanism 16 forming a portion of the facet joint broaching instrument 10 according to an exemplary embodiment of the present invention. FIG. 1 is a side view of the facet joint broaching instrument 10 of the present invention. The handles 12 are operable to receive a hand of a surgeon for use of the broaching instrument 10 to create a bore through a facet joint of the spine. The hinge 14 is operable to translate force applied by a surgeon through the handles 12 to the broaching mechanism 16. Specifically, the surgeon can open and close the handles 12 to actuate the broaching mechanism 16. The broaching mechanism 16 includes a pair of opposed arms 18, optionally coupled to a pair of opposed plates 20. In the exemplary embodiment illustrated, these opposed plates 20 are preferably substantially curved plates. The opposed plates 20 can also be substantially flat plates, etc. In operation, the opposed plates 20 are shaped and sized to securely engage the superior and inferior facets of a facet joint of the spine. For example, the handles 12 enable an applied force that is translated by the hinge 14 to open and close the opposing arms 18. Accordingly, the handles 12, the hinge 14, and the opposed arms 18 are constructed from a durable, rigid, and strong material capable of translating force to the broaching mechanism such that the force is capable to bore through the facet joint. For example, the various components above are constructed with constructions of surgical grade plastics or metals, such as titanium. Different dimensions of the various components are within the spirit and scope of the present invention.

One of the pair of opposed arms 18 is also coupled to a broaching spike 22, while the other of the pair of opposed arms includes a bore configured and positioned to receive excess bone material when the broaching mechanism 16 is actuated. The broaching spike 22 and the bore can also be coupled to the opposed plates 20. Each of the broaching spike 22 and the bore are detachably coupled to the opposed arms 18 and/or the opposed plates 20. The facet joint broaching instrument 10 is configured to bore the broaching spike 22 through the facet joint simultaneously pushing excess bone material from the boring operation through the superior and inferior facet through the bore thereby leaving a bore in the facet joint for stabilizing/immobilizing the facet joint with an implant described herein. Specifically, the broaching spike 22 includes a sharp point configured to penetrate through a bony structure, i.e. the facet joint, responsive to actuation of the broaching mechanism 16. Once the broaching spike 22 penetrates through the facet joint, the bolt 22 engages the bore at the opposed arm 18. Accordingly, the opposed arms 18 and/or the opposed plates 20 are positioned such that the broaching spike 22 and the bore are at the entrance/exit of the joint being immobilized. The surgeon can utilize the handles 12 to position the instrument 10 in the desired position.

After actuating the facet joint broaching instrument 10 and engaging the facet joint, the broaching spike 22 and the bore slidingly exit the facet joint while the instrument 10 is detached leaving the bore in the facet joint, i.e. by opening the handles 12 thereby slidingly disengaging the broaching spike 22 and the bore. The opposed arms 18 and the opposed plates 20 are configured in the broaching mechanism 16 to provide motion along a single axis, i.e. the hinge 14 and the opposed arms 18 ensure motion of the broaching spike 22 and the bore is constrained to the single axis. Thus, the broaching spike 22 engages the bore on the opposite opposed arm 18 through actuation of the broaching mechanism 16. Additionally, the broaching mechanism 16 includes a receiving chamber 24 configured and positioned to collect bone material that is broached from the facet joint of the spine and pushed through the bore by the broaching spike 22 when the broaching mechanism 16 is actuated. Specifically, the broaching spike 22 is configured to push out bone material as the broaching spike 22 is engaged through the joint, and the bone material is captured in the receiving chamber 24 and removed with the instrument 10.

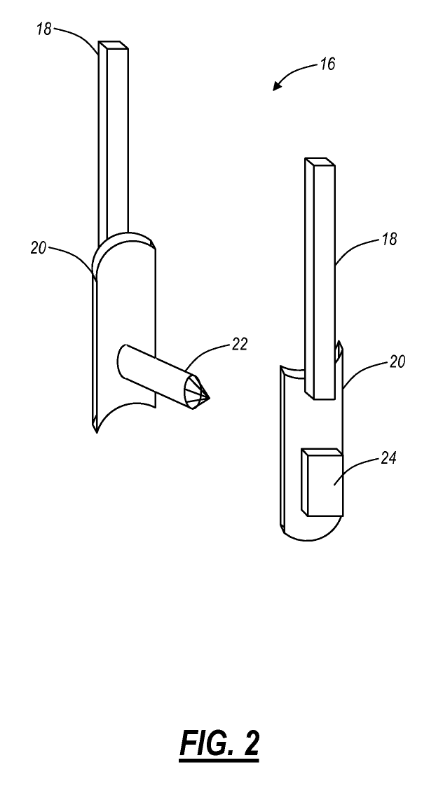

Referring to FIG. 2, an exploded perspective view illustrates the broaching mechanism 16 according to an exemplary embodiment of the present invention. In this exemplary embodiment, the opposed plates 20 are substantially curved plates with a curvature sufficient to surround a superior and inferior facet of a facet joint. Each of the opposed arms 18 are disposed to a separate opposed plate 20. The broaching spike 22 is fixedly coupled to a first opposed plate 20. The second opposed plate 20 includes the bore (not shown) and the receiving chamber 24. The bore in the opposed plate 20 is substantially dimensioned to receive the broaching spike 22, and the bore opens to the receiving chamber 24. Excess bone material from the broaching operation is deposited in the receiving chamber 24 through an opening in the opposed plate 20 and through an opening in the bore. Optionally, the receiving chamber 24 can be detachable from the opposed plate 20 to remove the excess bone material after use. Alternatively, the receiving chamber 24 can include an opening to remove the excess bone material after use.

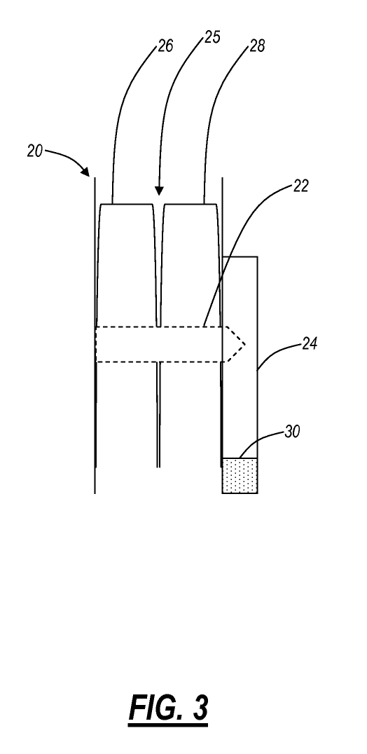

Referring to FIG. 3, an enlarged side view of a portion of the facet joint broaching instrument 10 of FIG. 1 is illustrated according to an exemplary embodiment of the present invention. Here, the facet joint broaching instrument 10 is engaging a facet joint 25 of the spine including a superior facet 26 and an inferior facet 28. The bone is illustrated schematically as the superior facet 26 and the inferior facet 28. FIG. 3 illustrates the facet joint 25 located between the opposed plates 20. The instrument 10 is surgically positioned such that the superior facet 26 and the inferior facet 28 are each adjacent to one of the opposed plates 20 in a desired position of the instrument 10 to effectuate immobilization and/or stabilization of the facet joint 25. FIG. 3 illustrates the broaching spike 22 fully engaged through each of the superior facet 26 and the inferior facet 28. As described above, this engagement results from an applied force to the broaching mechanism 16 that is translated to the opposed plates 20 thereby forcing the broaching spike 22 through the facets 26, 28 along an axis such that the opposed plates 20 fit the broaching spike 22 to the bore on the opposed plate 20. This engagement further results in bone material 30 being broached from the facet joint 25 of the spine and pushed through the bore (not illustrated) by the broaching spike 22 and collected in the receiving chamber 24 for removal with the instrument 10. The receiving chamber 24 can be detached from the instrument 10 and disposed of with the excess bone material 30. Further, another receiving chamber 24 can be attached to the instrument 10 for reuse in another operation.

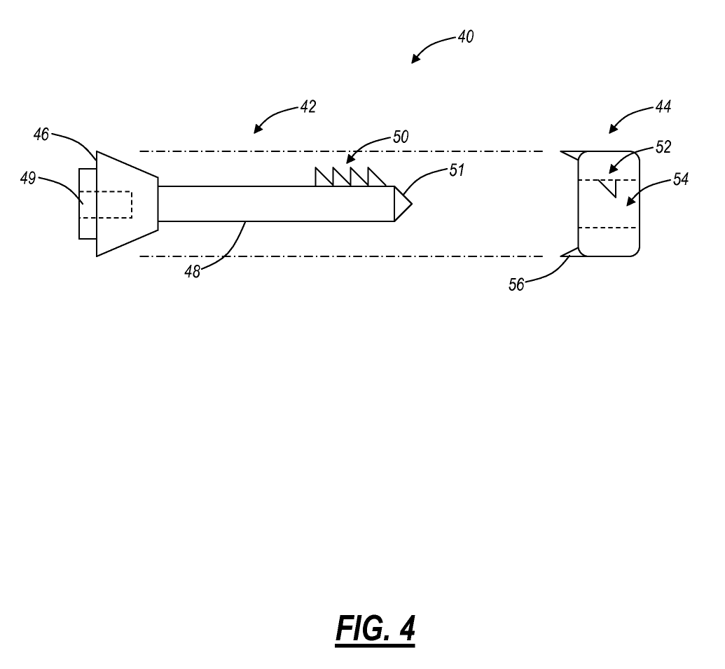

Referring to FIG. 4, an exploded side view of one exemplary embodiment of a facet joint broaching implant 40 of the present invention is illustrated. The facet joint broaching implant 40 includes a facet joint broaching bolt 42 and a facet joint broaching nut 44. The facet joint broaching bolt 42 includes a head portion 46 and an elongated shaft portion 48. The head portion 46 of the facet joint broaching bolt 42 is configured to selectively engage the facet joint broaching instrument 10 or a similar instrument without the broaching spike 22 and the receiving chamber 24, such as, for example, through a hollow channel 49 disposed within the head portion 46. The facet joint broaching instrument 10 or other instrument can include a pin or the like operable to engage the hollow channel 49. This engagement structure enables force to be translated from the facet joint broaching instrument 10 or other instrument to the broaching bolt 42 through the head portion 46 while enabling the broaching bolt 42 to detach from the facet joint broaching instrument 10 or other instrument. Accordingly, the facet joint broaching instrument 10 or other instrument is utilized to push the broaching bolt 42 through the bore created by the broaching spike 22. Other engagement structures are also contemplated.

The shaft portion 48 of the facet joint broaching bolt 42 includes a plurality of raised structures 50 that are configured to securely engage one or more raised structures 52 disposed within a bore 54 associated with the facet joint broaching nut 44. This engagement between the plurality of raised structures 50 and the raised structures 52 provides a locking mechanism between the broaching bolt 42 and the broaching nut 44. Additionally, this locking mechanism can be set to a desired position, i.e. on the plurality of raised structures 50, through the application of force through the instrument 10 or other instrument that inserts the broaching bolt 42 and the broaching nut 44 through the bore created by the broaching spike 22. Specifically, a surgeon can adjust the instrument 10 or other instrument until the raised structures 52 lock on a desired raised structure 50 on the broaching bolt 42. Additionally, the shaft portion 48 terminates in a pointed end 51 that is preferably sharp to enable entry through the bore in the facet joint 25 responsive to the applied force from the facet joint broaching instrument 10 or other instrument. The facet joint broaching nut 44 is also configured as detachably coupled to the facet joint broaching instrument 10 or other instrument, such as through a clamp, mount, or other engagement mechanism on the instrument 10 or other instrument. Preferably, the facet joint broaching nut 44 also includes a plurality of tooth structures 56 configured to frictionally engage a surface of the facet joint 25 of the spine. These tooth structures 56 are preferably sharp such that they engage into the facet joint 25 responsive to applied force from the instrument 10 or other instrument thereby further locking the facet joint broaching implant 40.

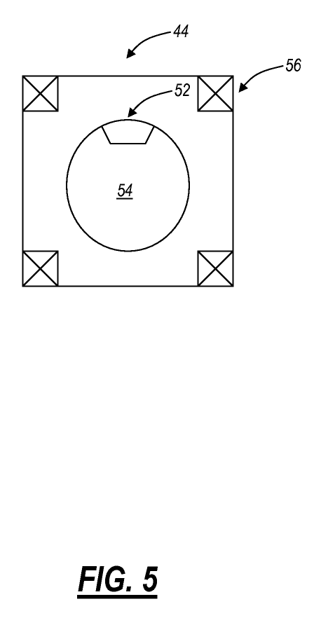

Referring to FIG. 5, an enlarged end view of the facet joint broaching nut 44 of FIG. 3 is illustrated according to an exemplary embodiment of the present invention. The bore 54 is preferably open at both ends to allow the excess bone material from the broaching operating to enter the receiving chamber 24. Additionally, the bore 54 covers substantially all of the pointed end 51 thereby protecting surrounding tissue, muscle, nerves, and the like. In this exemplary embodiment, the facet joint broaching nut 44 includes a substantially square shape with the bore 54 including a cylindrical shape. Other shapes are also contemplated by the present invention. The square shape advantageously can be easily coupled and detached from the instrument 10, such as through a mount or the like.

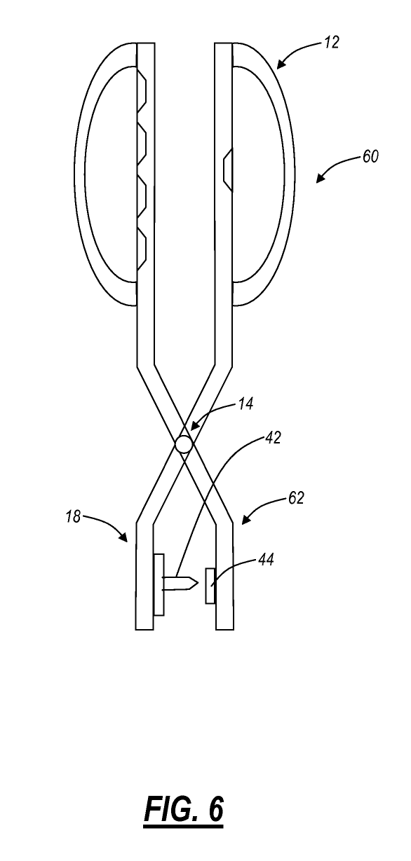

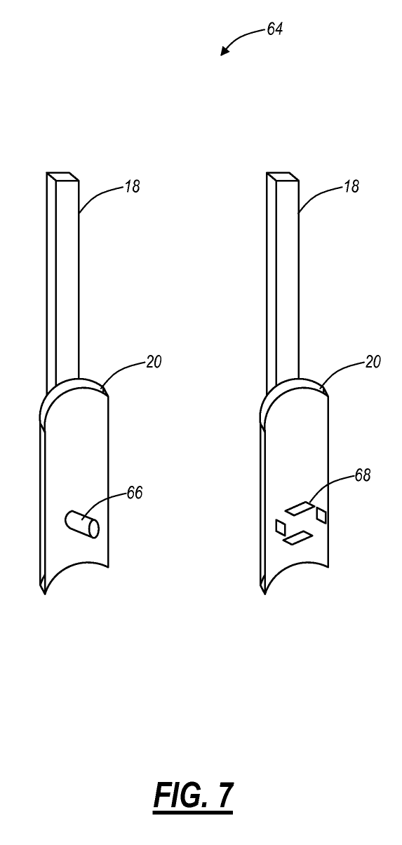

Referring to FIG. 6, a facet joint implant instrument 60 includes a pair of handles 12 that are connected via a hinge 14 and operable for actuating an implanting mechanism 62 according to an exemplary embodiment of the present invention. FIG. 6 is a side view of the facet joint implant instrument 60 of the present invention. The implant instrument 60 is similar to the facet joint broaching instrument 10 of FIG. 1 without the broaching spike 22 and the receiving chamber 24. The facet joint implant instrument 60 is operable to implant the facet joint broaching implant 40 of FIG. 4 in a bore created by the facet joint broaching instrument 10 of FIG. 1. The implant instrument 60 includes an engagement mechanism 64 (illustrated in FIG. 7) for engaging the broaching bolt 42 and the broaching nut 44. In an exemplary embodiment, the facet joint implant instrument 60 is the same as the facet joint broaching instrument 10 of FIG. 1 with the broaching spike 22 and the receiving chamber 24 removed. The handles 12, the hinge 14, and the opposed arms 18 are constructed from a durable, rigid, and strong material capable of translating force to the broaching mechanism such that the force is capable to slide the implant 40 through the facet joint. For example, the various components above are constructed with constructions of surgical grade plastics or metals, such as titanium. Different dimensions of the various components are within the spirit and scope of the present invention.

Referring to FIG. 7, an exploded perspective view illustrates an engagement mechanism 64 on the facet joint implant instrument 60 according to an exemplary embodiment of the present invention. FIG. 7 illustrates two opposed plates 20 each disposed to an opposed arm 18. For the facet joint broaching bolt 42, the opposed plate 20 includes a pin 66 dimensioned to engage the hollow channel 49 in the head portion 46 of the facet joint broaching bolt 42. The pin 66 enables the plate 20 to disengage from the facet joint broaching bolt 42 by opening the instrument 60 and to translate force to the facet joint broaching bolt 42 by closing the instrument 60. Specifically, the instrument 60 can operate on a single linear axis thereby engaging and disengaging the facet joint broaching bolt 42 through the pin 66. Other engagement structures are also contemplated for the facet joint broaching bolt 42. For the facet joint broaching nut 44, the opposed plate 22 includes two or more notches 68 for detachably coupling the nut 44 to the instrument. In FIG. 7, the two or more notches 68 are illustrated with four notches to receive a substantially square nut. For other shaped nuts, the present invention contemplates other shapes for the notches 68, such as, for example, a single circular notch to receive a substantially circular nut. Similar to the pin 66, the notches 68 enable the instrument 60 to move the nut 44 along the single linear axis to engage the bolt 42 and to disengage from the instrument 60.

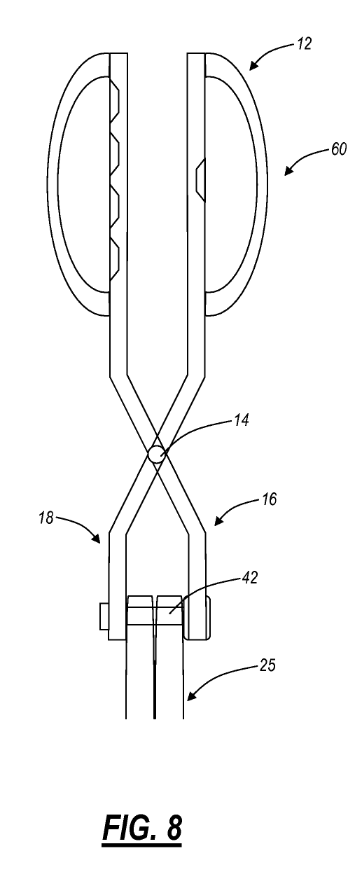

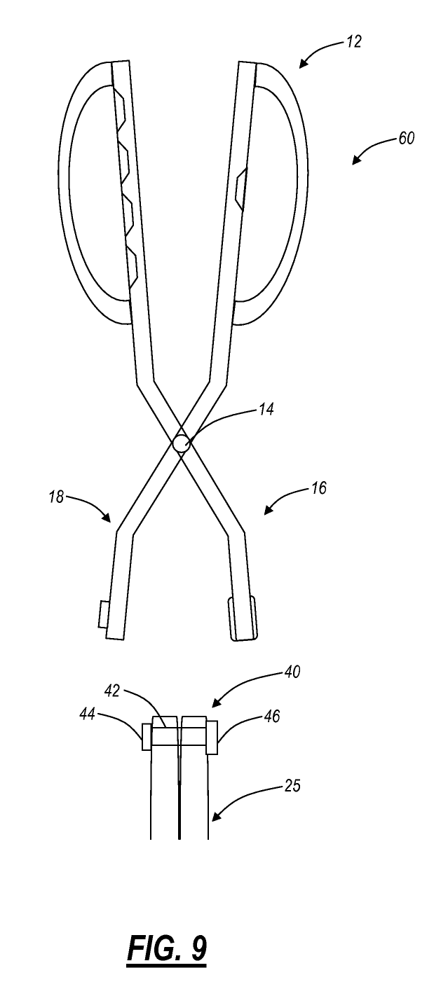

Referring to FIGS. 8 and 9, side views illustrate the facet joint implant instrument 60 of FIG. 6 engaging the facet joint broaching implant 40 of FIG. 4 through a facet joint 25 according to an exemplary embodiment of the present invention. FIG. 7 illustrates the instrument 10 fully engaging the implant 40 through the facet joint 25. FIG. 8 illustrates the instrument 10 disengaged from the implant 40 leaving the implant 40 engaged to the facet joint 25 through the broaching nut 44 and the head portion 46 of the broaching bolt 22. In FIG. 8, the instrument 10 removes the excess bone material in the receiving chamber 24.

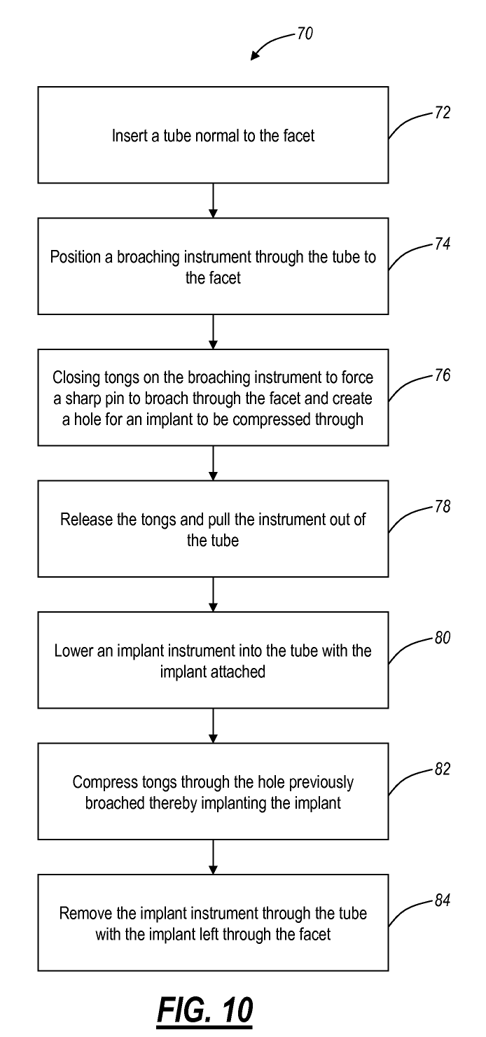

Referring to FIG. 10, a flow chart illustrates implantation method 70 utilizing the facet joint broaching instrument 10 of FIG. 1, the facet joint broaching implant 40 of FIG. 4, the facet joint implant instrument 60 of FIG. 6, or the like according to an exemplary embodiment of the present invention. An access tube is inserted normal to a facet joint of the spine (step 72). For example, the access tube can include a 23 mm access tube or the like. This approach is similar to a transforaminal approach, for example. The facet joint broaching instrument 10 is then positioned through the access tube adjacent to a superior and inferior facet of the facet joint (step 74). Tongs on the broaching instrument are closed causing a sharp pin to broach through the facet and create a hole for an implant to be compressed through (step 76). For example, this can include compressing a pair of handles to actuate a broaching mechanism as described herein. A broaching spike on the broaching instrument broaches the facet joint of the spine and creates a hole for a facet joint broaching implant to be disposed through. The tongs are released and the broaching instrument is pulled out of the tube (step 78). For example, the pair of handles can be released to deactuate the broaching mechanism and the facet joint broaching instrument is removed from the access tube. The same broaching instrument or a separate implant instrument is positioned in the access tube with an implant attached (step 80). Tongs on the instrument are compressed engaging the implant through the hole previously broached thereby implanting the instrument (step 82). The instrument is removed through the tube with the implant left through the facet joint thereby providing stabilization and immobilization of the facet joint (step 84).

Although the present invention has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present invention and are intended to be covered by the following claims.

What is claimed is:

1. A facet joint broaching instrument and implant for stabilizing/immobilizing a facet joint of the spine via an open or minimally-invasive surgical procedure, comprising:
a broaching mechanism comprising a pair of opposed arms, wherein one of the pair of opposed arms is coupled to a broaching spike and the other of the pair of opposed arms defines a bore configured and positioned to receive the broaching spike; and
an actuation mechanism operable for selectively compressing the pair of opposed arms of the broaching mechanism together and retracting the pair of opposed arms of the broaching mechanism apart.

2. The facet joint broaching instrument and implant of claim 1, wherein the actuation mechanism comprises a pair of handles connected via a hinge and coupled to the broaching mechanism.

3. The facet joint broaching instrument and implant of claim 1, further comprising a pair of opposed plates coupled to the pair of opposed arms, wherein the pair of opposed plates are shaped and sized to securely engage a superior facet and an inferior facet of a facet joint of a spine.

4. The facet joint broaching instrument and implant of claim 1, further comprising a receiving chamber coupled to the other of the pair of opposed arms, wherein the receiving chamber is configured and positioned to collect bone material that is broached from a facet joint of a spine and pushed through the bore by the broaching spike when the broaching mechanism is actuated.

5. The facet joint broaching instrument and implant of claim 1, further comprising, with or without the broaching spike removed, a facet joint broaching implant comprising a facet joint broaching bolt selectively engaging the one of the pair of opposed arms and a facet joint broaching nut selectively engaging the other of the pair of opposed arms.

6. The facet joint broaching instrument and implant of claim 5, wherein the facet joint broaching bolt comprises a head portion and a shaft portion, wherein the head portion of the facet joint broaching bolt is configured to selectively engage the one of the pair of opposed arms and the shaft portion of the facet joint broaching bolt comprises a plurality of raised structures that are configured to securely engage one or more raised structures disposed within a bore associated with the facet joint broaching nut, and wherein the facet joint broaching nut is configured to selectively engage the other of the pair of opposed arms.

7. The facet joint broaching instrument and implant of claim 5, wherein the facet joint broaching nut comprises a plurality of tooth structures configured to frictionally engage a surface of a facet joint of a spine.

8. A facet joint broaching method for stabilizing/immobilizing a facet joint of the spine via an open or minimally-invasive surgical procedure, comprising:
providing a broaching mechanism comprising a pair of opposed arms, wherein one of the pair of opposed arms is coupled to a broaching spike and the other of the pair of opposed arms defines a bore configured and positioned to receive the broaching spike; and
providing an actuation mechanism operable for selectively compressing the pair of opposed arms of the broaching mechanism together and retracting the pair of opposed arms of the broaching mechanism apart.

9. The facet joint broaching method of claim 8, wherein the actuation mechanism comprises a pair of handles connected via a hinge and coupled to the broaching mechanism.

10. The facet joint broaching method of claim 8, further comprising providing a pair of opposed plates coupled to the pair of opposed arms, wherein the pair of opposed plates are shaped and sized to securely engage a superior facet and an inferior facet of a facet joint of a spine.

11. The facet joint broaching method of claim 8, further comprising providing a receiving chamber coupled to the other of the pair of opposed arms, wherein the receiving chamber is configured and positioned to collect bone material that is broached from a facet joint of a spine and pushed through the bore by the broaching spike when the broaching mechanism is actuated.

12. The facet joint broaching method of claim 8, further comprising, with or without the broaching spike removed, providing a facet joint broaching implant comprising a facet joint broaching bolt selectively engaging the one of the pair of opposed arms and a facet joint broaching nut selectively engaging the other of the pair of opposed arms.

13. The facet joint broaching method of claim 12, wherein the facet joint broaching bolt comprises a head portion and a shaft portion, wherein the head portion of the facet joint broaching bolt is configured to selectively engage the one of the pair of opposed arms and the shaft portion of the facet joint broaching bolt comprises a plurality of raised structures that are configured to securely engage one or more raised structures disposed within a bore associated with the facet joint broaching nut, and wherein the facet joint broaching nut is configured to selectively engage the other of the pair of opposed arms.

14. The facet joint broaching method of claim 12, wherein the facet joint broaching nut comprises a plurality of tooth structures configured to frictionally engage a surface of a facet joint of a spine.

15. The facet joint broaching method of claim 12, wherein the broaching mechanism is selectively disposed about a facet joint of a spine through an access tube.

16. A facet joint implantation method for stabilizing/immobilizing a facet joint of the spine via an open or minimally-invasive surgical procedure, comprising:
inserting an access tube normal to a facet joint of a patient;
positioning a broaching instrument through the access tube adjacent to a superior and inferior facet of the facet joint;
closing tongs on the broaching instrument to force a sharp pin to broach through the superior and inferior facet to create a hole;
releasing the tongs;
pulling the broaching instrument out of the tube;
positioning an implant instrument in the access tube with an implant attached;
closing tongs on the implant instrument to compress the implant through the hole previously broached; and
removing the implant instrument through the access tube with the implant left through the facet joint.

17. The facet joint implantation method of claim 16, further comprising:
pushing excess bone material through the hole while the sharp pin is broached through the superior and inferior facet; and
collecting the excess bone material in a receiving chamber of the broaching instrument.

18. The facet joint implantation method of claim 16, further comprising:
adjusting a nut of the implant to a desired position to lock the implant to the facet joint.

19. The facet joint implantation method of claim 17, wherein the broaching instrument and the implant instrument comprise a single instrument with the sharp pin, the receiving chamber, and the implant detachable from the single instrument.
